(19) United States
(12) Patent Application Publication
(10) Pub. No.: US 2008/0269735 A1
(43) Pub. Date: Oct. 30, 2008

(54) Optical array for treating biological tissue

(76) Inventors: Agustina Vila Echague, Milton, MA (US); Jayant D. Bhawalkar, Brighton, MA (US); James C. Hsia, Weston, MA (US); Paul R. Lucchese, Sudbury, MA (US)

Correspondence Address: PROSKAUER ROSE LLP, ONE INTERNATIONAL PLACE, BOSTON, MA, 02110, US

(21) Appl. No.: 11/796,146

(22) Filed: Apr. 26, 2007

(51) Int. Cl. A61B 18/18

(52) U.S. Cl. 606/15

(57) ABSTRACT

An apparatus can treat biological tissue using a base member, a plurality of needles, and a plurality of fiber optics. The plurality of needles extend from the base member. Each needle defines a bore capable of receiving a fiber optic and has an end. The plurality of needles form an array capable of penetrating a biological tissue and positioning each end within a subsurface volume of the biological tissue. Each fiber optic is adapted for insertion into the bore of each needle, and each fiber optic is capable of delivering electromagnetic radiation to the subsurface volume of the biological tissue to treat the biological tissue.

### FIELD OF THE INVENTION

The invention relates generally to apparatus and methods for treating biological tissue using electromagnetic radiation. In particular, the invention relates to an optical array for treating biological tissue.

### BACKGROUND OF THE INVENTION

Liposuction can sculpt the human body by removing unwanted subcutaneous fatty tissue. Local removal of unwanted subcutaneous fatty tissue, and the corresponding improvement of body shape, can be a strong reinforcement for behavioral modifications related to diet and exercise, which reduce obesity and related diseases. For these reasons, liposuction is the most popular cosmetic surgery performed in the United States. According to the American Society of Aesthetic Plastic Surgery, 324,000 liposuctions and 135,000 abdominoplasties were performed in 2005. However, liposuction is risky and has a mortality rate between about 20 and about 100 deaths per 100,000 procedures. Additional complications can include adverse reactions to anesthesia, embolisms, organ perforations, infections, and post operative pain.

The demand for and the risks of traditional liposuction suggest a need for a minimally invasive alternative to liposuction and abdominoplasty surgery that will minimize mortality, adverse side effects, and post operative recovery time. Laser and other light based devices present alternatives to traditional liposuction. For example, one technique employs a laser designed to remove excess fatty tissue by selective interaction of adipocytes. This technique may result in disintegration of the membranes of the adipocytes and the elimination of their contents, either naturally or by aspiration. The technique employs a laser in conjunction with a small cannula. Nevertheless the risks of mortality and complications for this technique can be similar to traditional liposuction.

### SUMMARY OF THE INVENTION

The invention, in various embodiments, provides methods and apparatus for treating biological tissue. The biological tissue can be, but is not limited to, skin and hypodermal features such as subcutaneous fatty tissue. The methods can offer alternatives to traditional liposuction. The apparatus can include an array of needles to penetrate the biological tissue and fiber optics to deliver electromagnetic radiation to a subsurface volume of the biological tissue to treat the biological tissue. In some embodiments, a treatment can melt subcutaneous fatty tissue and remove the resulting melted and/or liquefied tissue by suction or drainage. A treatment can also contour and/or remodel biological tissue. Advantages include minimizing the amount of free fatty acids left inside the body, which can minimize post-operative side effects such as embolism. Other advantages include reducing or eliminating localized lipodystrophy, which can minimize or eliminate post-operative flaccidity by localized laser heating, and reductions in mortality. Additional advantages over traditional liposuction include reduced eliminating the need for an incision and reduced trauma, embolisms, organ perforations, infections, and post operative pain. Further advantages include reducing or eliminating the need for anesthesia, and thus adverse reactions to anesthesia, as well as reducing recovery time.

By applying electromagnetic radiation to subcutaneous fatty tissue through a minimally invasive array of needles, the epidermis and the dermis can be spared from injury from the electromagnetic radiation. Furthermore, the electromagnetic radiation can diffuse within the subcutaneous fatty tissue, to effect a homogeneous treatment. Lower powers can also be used because the electromagnetic radiation is delivered directly to the fatty tissue and does not need to travel through the epidermis and/or dermis. At east a portion of the subcutaneous fatty tissue can melt and/or liquefy, and fibrosis and/or tightening of the skin can result without scarring the epidermis and/or dermis. Additionally, subcutaneous fatty tissue and/or melted subcutaneous fatty tissue can be suctioned or otherwise removed to mitigate the side effects of traditional liposuction. Subcutaneous fatty tissue can be removed in smaller and/or more controlled amounts than traditional liposuction to further mitigate the side effects of traditional liposuction. For example, when less fatty tissue is removed, the body may not respond by regenerating fatty tissue, as can be the case with traditional liposuction.

In one aspect, the invention features an apparatus for treating biological tissue including a base member, a plurality of needles, and a plurality of fiber optics. The plurality of needles extends from the base member. Each needle defines a bore capable of receiving a fiber optic and has an end. The plurality of needles form an array capable of penetrating a biological tissue and positioning each end within a subsurface volume of the biological tissue. Each fiber optic is adapted for insertion into the bore of each needle and each fiber optic is capable of delivering electromagnetic radiation to the subsurface volume of the biological tissue to treat the biological tissue.

In another aspect, the invention features an apparatus for treating biological tissue including a base member, a first needle, a second needle, a first fiber optic, and a second fiber optic. The first needle extends from the base member, defines a first bore, and has a first end. The second needle extends from the base member and is spaced from the first needle. The second needle defines a second bore and has a second end. The first needle and the second needle form an array of needles capable of penetrating a biological tissue and positioning the first end and the second end within a subsurface volume of the biological tissue. The first fiber optic is adapted for insertion into the first bore and the second fiber optic adapted for insertion into the second bore. The first fiber optic and the second fiber optic are capable of delivering electromagnetic radiation to the subsurface volume of the biological tissue to treat the biological tissue.

In still another aspect, the invention features a method for treating biological tissue including penetrating a surface of a biological tissue with a plurality of needles. Each needle defines a bore capable of receiving a fiber optic and has an end. The method also includes positioning each end within a subsurface volume of the biological tissue and delivering electromagnetic radiation through a plurality of fiber optics to the subsurface volume of the biological tissue to treat the biological tissue. At least one fiber optic of the plurality of fiber optics is inserted within the bore of each needle.

In yet another aspect, the invention features a method for treating biological tissue including penetrating a surface of a biological tissue with a plurality of waveguides. Each waveguide has an end. The method also includes positioning each end within a subsurface volume of the biological tissue and delivering electromagnetic radiation through the plurality of waveguides to the subsurface volume of the biological tissue to treat the biological tissue.

In still yet another aspect, the invention features an apparatus for treating biological tissue comprising a plurality of waveguides extending from a base member. Each waveguide has an end. The plurality of waveguides forms an array capable of penetrating a biological tissue and positioning each end within a subsurface volume of the biological tissue. The plurality of waveguides can deliver electromagnetic radiation to the subsurface volume of the biological tissue to treat the biological tissue.

In other examples, any of the aspects above, or any apparatus or method described herein, can include one or more of the following features.

In various embodiments, the apparatus can include a source of a beam of electromagnetic radiation. The source of the beam of electromagnetic radiation can be a laser, a light emitting diode, an incandescent lamp, a flash lamp, or a gas discharge lamp. In one embodiment, each fiber optic employs free-space coupling to deliver the beam of electromagnetic radiation to the biological tissue. The fiber optic can be adapted to be movable within the bore, extendable beyond the end, and retractable into the bore. The fiber optic can include sapphire.

In some embodiments, the apparatus can include a beam of electromagnetic radiation characterized by a wavelength between about 400 nanometers and about 10,600 nanometers. The beam of electromagnetic radiation can have a power between about 0.1 watts and about 500 watts. The beam of electromagnetic radiation can have a pulse duration between about 0.1 microseconds and about 10 seconds.

In certain embodiments, the apparatus can include a needle adapted for penetrating biological tissue to a depth of about 1.5 to about 30 mm from a surface of the biological tissue. Each needle can have a diameter between about 0.2 mm and about 2 mm. In one embodiment, each needle has a diameter of less than about 1 millimeter.

In various embodiments, the apparatus can include a means for suctioning at least a portion of the biological tissue.

In some embodiments, the apparatus can include a means for cooling at least a portion of the biological tissue.

In certain embodiments, the apparatus can include a means for mitigating pain in at least a portion of the biological tissue.

In various embodiments, the apparatus can include a scanner for translating or rotating the base member.

In some embodiments, the method includes penetrating the surface of the biological tissue with the plurality of needles forms an angle of about 45 degrees and about 90 degrees between the surface of the biological tissue and each needle.

In certain embodiments, the method can include applying suction to the subsurface volume of the biological tissue.

In various embodiments, the method can include cooling at least a portion of the biological tissue.

In some embodiments, the method can include mitigating at least a portion of pain or discomfort.

In certain embodiments, the method can include moving a portion of the at least one fiber optic within the subsurface volume of biological tissue while delivering electromagnetic radiation.

In various embodiments, the method can include: (i) removing each end from the subsurface volume of the biological tissue; (ii) translating or rotating the plurality of needles relative to the biological tissue; (iii) penetrating the surface of the of biological tissue with the plurality of needles; (iv) positioning the each end within a second subsurface volume of the biological tissue; and/or (v) delivering electromagnetic radiation through each fiber optic inserted within the bore to the second subsurface volume of the biological tissue to treat the biological tissue. In one embodiment, the method can include inserting each fiber optic in the bore.

Other aspects and advantages of the invention will become apparent from the following drawings and description, all of which illustrate principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIGS. 1A-1C illustrate an exemplary apparatus having a base member and a plurality of needles and fiber optics for treating biological tissue.

FIGS. 2A-2C illustrate exemplary fiber optic tips.

FIGS. 3A-3B illustrate an exemplary needle with a fiber optic and a vacuum.

FIG. 4 illustrates an exemplary apparatus having a base member and a plurality of needles for treating biological tissue.

FIGS. 5A-5B illustrate exemplary fiber optic systems.

FIGS. 6A-6C illustrate a method for treating biological tissue.

FIG. 7 illustrates another method for treating biological tissue.

FIG. 8 illustrates still another method for treating biological tissue.

FIG. 9 illustrates yet another method for treating biological tissue.

FIGS. 10A-10B show an exemplary region of treated skin.

FIGS. 11A-11B show another exemplary region of treated skin.

FIGS. 12A-12C illustrate an exemplary apparatus having a base member and a plurality of waveguides for treating biological tissue.

FIG. 13 shows an absorbent pad including an absorbent material.

FIGS. 14A-14B show a region of skin treated with a puncturing device.

FIGS. 15A-15B show a puncturing device with an absorbent pad.

### DETAILED DESCRIPTION OF THE INVENTION

A plurality of waveguides formed in an array pattern can be inserted into biological tissue. The waveguides can be positioned in the tissue so that a subsurface volume of the biological tissue can be treated. Electromagnetic radiation is delivered using the waveguides to treat the subsurface volume.

In certain embodiments, a treatment can be for one or more of the following indications: acne, erythema, fat, cellulite, oily skin, pigmented lesions, pores, scarring, vascular lesions (including port wine stains), and wrinkles, as well as for skin rejuvenation, hair removal, and hair regrowth. Target chromophores can include water, fat, collagen, blood or a blood component, melanin, or other commonly targeted skin chromophores in cosmetic and dermatologic treatments.

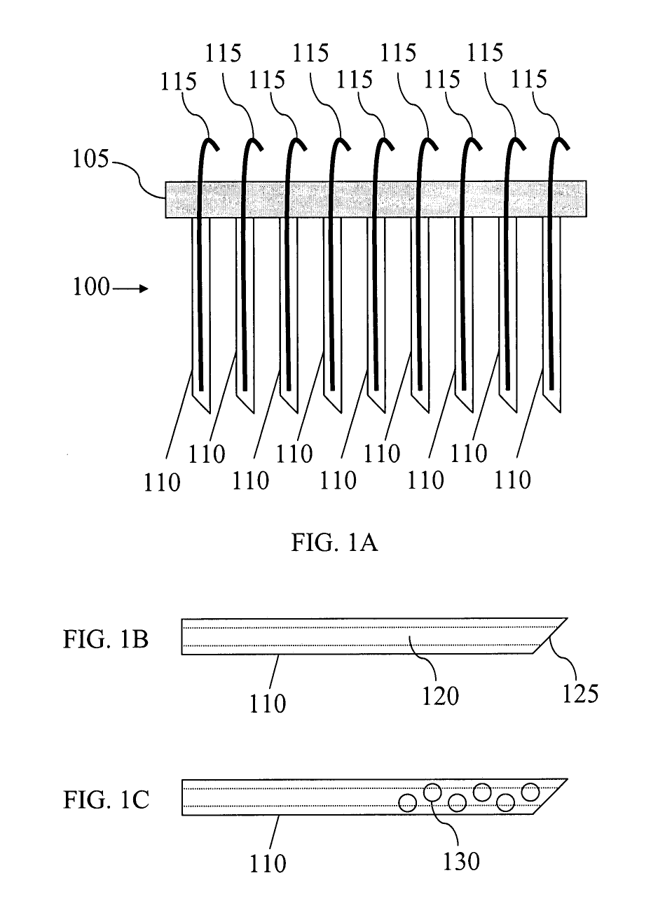

FIG. 1A illustrates an apparatus 100 for treating biological tissue including a base member 105, a plurality of needles 110 extending from the base member 105, and a plurality of fiber optics 115. The base member 105 can be made from a metal, plastic, or polymer material. The plurality of needles 110 can be attached to the base member 105, or can be removable. The base member 105 can be flexible, which can allow the plurality of needles 110 extending from the base member 105 to match a contour of the biological tissue.

FIG. 1B illustrates a needle 110 in detail. The needle 110 defines a bore 120 capable of receiving a fiber optic 115 and has an end 125.

FIG. 1C illustrates another embodiment of a needle 110 in detail. The needle 110 can define one or more openings 130 that allow electromagnetic radiation to radiate from the needle 110 from a region other than about the end 125. The one or more openings 130 can facilitate simultaneous treatment at more than one depth within the biological tissue.

In various embodiments, each needle 110 is adapted for penetrating biological tissue to a depth of about 1.5 to about 30 mm from a surface of the biological tissue. A needle 110 can be adapted to penetrate biological tissue to a depth of about 0.5 to about 2 cm. In certain embodiments, a needle 110 can be adapted to penetrate biological tissue to a depth of up to about 1 cm or about 2 cm. The diameter of each needle can be between about 0.2 mm and about 2 mm. In one embodiment, the diameter of each needle 110 is less than about 1 millimeter. In various embodiments, each needle 110 can be a different diameter and/or length. This can result in each needle 110 being positioned at a different depth within the subsurface volume of biological tissue, and can facilitate treatment at more than one depth. Variations in needle 110 length can also facilitate simultaneously treatment of a larger volume of biological tissue. Each needle 110 can be disposable. The base member 105 can be disposable. In one embodiment, the base member 105 and plurality of needles 110 can be a disposable, and/or can be a cartridge. Alternatively, the waveguide 1010 and/or base member 1005 can be sterilized and reusable. Each needle 110 can include stainless steel or aluminum, and can be a 30G needle or a 27G needle. In one embodiment, the needle 110 can be a STERIJECT® Rimos or Mesoram needle, which can be used as a multiinjector for mesotherapy.

The plurality of needles 110 form an array capable of penetrating a biological tissue and positioning each end 125 within a subsurface volume of the biological tissue. The base member 105 can function as a depth gauge by limiting the depth to which a needle 110 can be inserted into the biological tissue. The base member 105 and the needle 110 can be adjustable, so that the length of the needle 110 extending from the base member 105 can be adjusted. In one embodiment, the array of needles 110 are passed through holes in a rigid frame or a base member 105 and epoxied or fused to the frame or a base member 105. A biocompatible epoxy or low temperature glass flit can be used to epoxy or fuse the needles 110. Each fiber optic 115 is adapted for insertion into the bore 120 of each needle 110, and each fiber optic 115 is capable of delivering electromagnetic radiation to the subsurface volume of the biological tissue to treat the biological tissue.

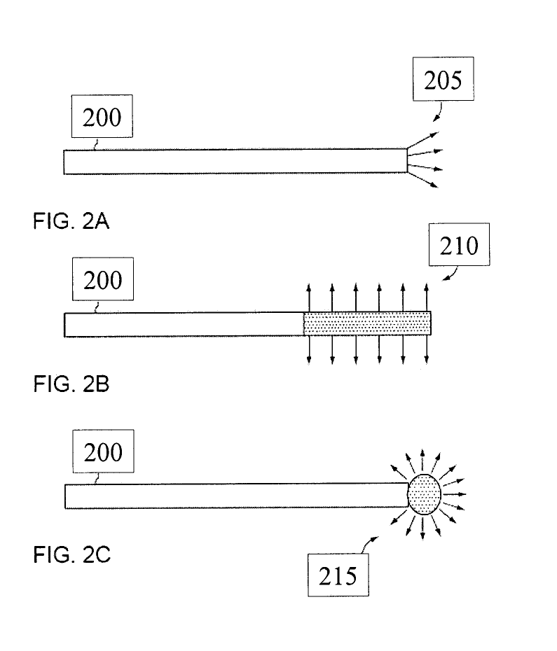

FIGS. 2A-2C illustrate exemplary fiber optic tips. In various embodiments, non-diffusing fiber optic tips can direct electromagnetic radiation substantially along the longitudinal axis of the fiber optic 200 to deliver the light to the biological tissue. In other embodiments, diffusing fiber tips can be used to deliver electromagnetic radiation to the biological tissue. Using diffusing fiber optic tips, electromagnetic radiation can be directed laterally from an end portion of the fiber optic 200, which can allow more precise heating and injury of the biological tissue and provide a more uniform and predictable treatment of the biological tissue. Furthermore, means known in the art can also be used to manipulate the end portion of the fiber optic 200. For example, the fiber optic 200 can be attached to a guide that can be manually or mechanically manipulated. The fiber optic can be adapted to be movable within the bore, extendable beyond the end of the needle, and/or retractable into the bore. The fiber optic can include sapphire. For example, the fiber optic or fiber optic tip can be sapphire.

FIG. 2A illustrates a fiber optic 200 with a bare fiber tip 205. The bare fiber tip 205 can be the simplest and least expensive design, and can be obtained by cleaving a fiber optic. In one embodiment, the fiber optic has a diameter of about 300 microns. For example, the fiber optic 200 can be a fiber optic manufactured or sourced from SCHOTT North America, Inc., which can cover a broad spectral range. The fiber optic 200 can be of diameter about 30 μm, 50 μm, 70 μm or a different custom diameter. The arrows approximate the propagation of electromagnetic radiation from the bare fiber tip 205.

FIG. 2B illustrates a fiber optic 200 with a linear diffuser tip 210. The light from the diffuser tip 210 is delivered laterally from the fiber optic 200 to the biological tissue. FIG. 2C illustrates a fiber optic 200 with a spherical ball-type diffuser tip 215, which emits light radially from the fiber tip. The diffuser tips 210 and 215 can include a scattering material, such as a polymer cover or a ceramic cover. The scattering material can overcome the index of refraction matching properties of the fiber optic and the adjacent fluid or biological tissue. The diffuser tips 210 and 215 are more expensive than bare fiber tip 205, but may provide better control of the light delivered. In various embodiments, the diffuser tip 210 or 215 can be permanently or removably affixed to the fiber optic 200. The diffuser tips 210 or 215 can be affixed using an adhesive, a bonding agent, a joining compound, an epoxy, a clip, a thread, other suitable mechanical connection or attachment means, or some combination thereof.

In various embodiments, the invention can include additional features to facilitate treatment of the biological tissue. For example, the apparatus can include a means for suctioning at least a portion of the biological tissue. The means of suctioning can be a needle 110 and a vacuum, or can be a different type of needle or tube, to remove and/or drain at least a portion of the subsurface volume of biological tissue.

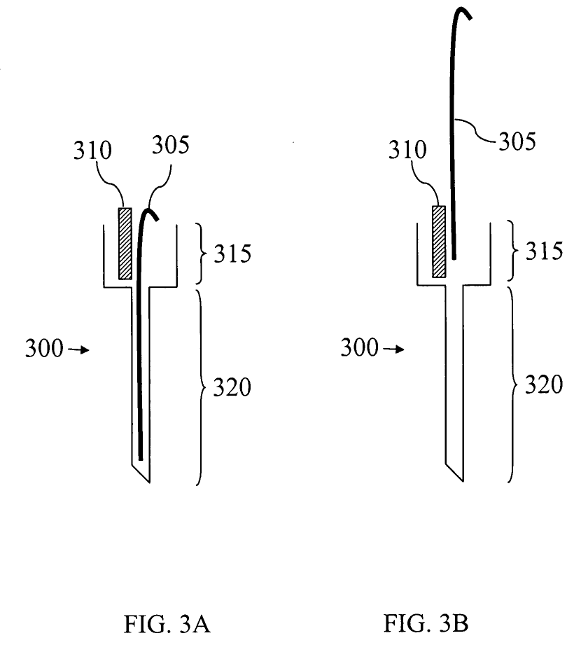

FIG. 3A-3B illustrate an exemplary needle 300 with a fiber optic 305 and a vacuum 310 for suctioning at least a portion of the biological tissue. The needle 300 can have two or more dimensions. For example, the needle 300 can have a first 315 portion of a first diameter positioned adjacent a base member, and a second 320 portion extending from the base member. The first 315 portion can affix the needle 300 to the base member, receive the fiber optic 305, and include the vacuum 310. The second 320 portion can penetrate a biological tissue, facilitate delivery of the fiber optic 305 to a subsurface volume of the biological tissue, and facilitate use of the vacuum 310 for suctioning at least a portion of the subsurface volume of biological tissue. The vacuum 310 can be used for suctioning fatty tissue after it is melted and/or liquefied by electromagnetic radiation delivered by the fiber optic 305. FIG. 3B illustrates an arrangement where the fiber optic 305 is withdrawn from the second 320 portion before employing the vacuum 310 for suctioning fatty tissue. Employing a needle with a fiber optic that can be retracted to allow suctioning can result in a needle with a smaller diameter.

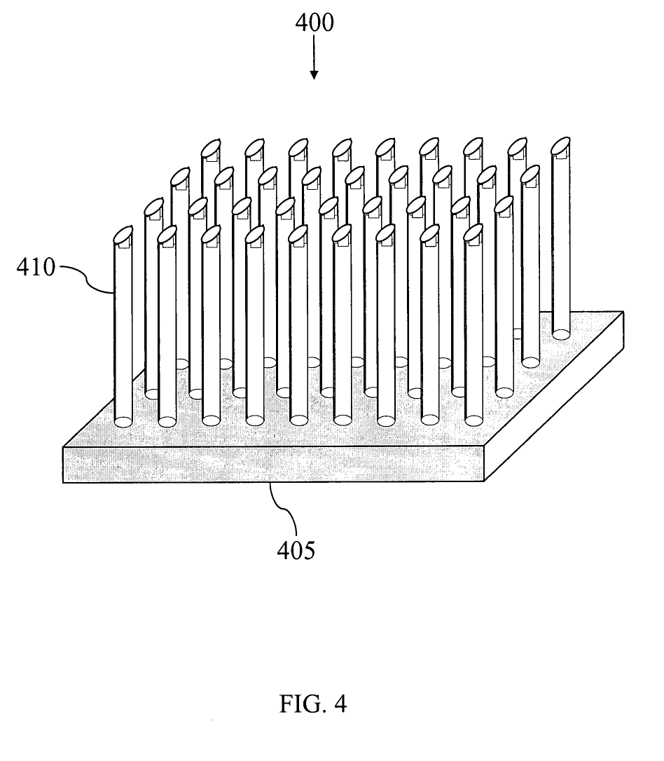

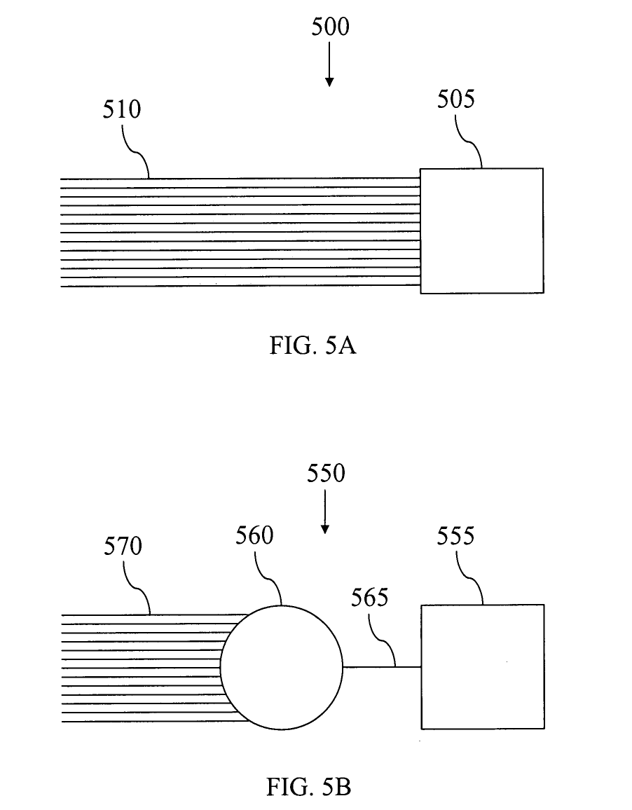

FIG. 4 illustrates another view of an apparatus 400 for treating biological tissue including a base member 405, a plurality of needles 410, and a plurality of fiber optics (not shown). The plurality of needles 410 can include the same features as the plurality of needles 110 described in FIG. 1. The apparatus 400 illustrates a regular, two dimensional array of the plurality of needles 410.

The invention is not limited to the number and/or arrangement of needles shown in FIGS. 1 and 4. For example, the invention includes apparatuses with regular and irregular, as well as one and two dimensional, arrays of two or more needles. Furthermore, the invention includes embodiments where the needle does not form a right angle with the base member. For example, the invention includes apparatuses where the plurality of needles forms an angle of about 45 degrees, or any other angle between about 30 degrees and about 90 degrees between the base member and each needle, to facilitate nonperpendicular entry into the biological tissue.

In various embodiments, the base member and/or needle array can have a diameter of about 10 cm, or dimensions up to about 10 by 10 cm, 5 by 5 cm, or 5 by 10 cm. The base member and/or needle array can be square, rectangular, circular, ovoid, or polygonal. Polygonal base members can be used for “tiling,” to cover a larger area by forming a regular pattern of individual treatment areas. In various embodiments individual needles can be spaced less than about 5 mm apart or between about 50 microns to about 2 mm apart. In some embodiments, individual needles can be spaced between about 500 microns to about 1 mm apart. In certain embodiments, needles are spaced about 0.5 mm or about 1 mm apart. The spacing between needles in an array need not be uniform, and can be closer in areas where a greater amount of damage or more precise control of damage in the target area of tissue is desired. In one embodiment, the array of needles can include pairs of needles separated from adjacent pairs by larger distances. Needles can be arranged in a regular or near-regular square, triangular, or other geometrical arrays. The pattern of damage and/or tissue reshaping can be controlled by adjusting the intensity and/or duration of power transmitted to individual fiber optics. An array of needles can distribute pressure over a larger area when puncturing the skin, to reduce pain and/or discomfort.

FIG. 5A illustrates an exemplary fiber optic system 500 including a source 505 of electromagnetic radiation and a plurality of fiber optics 510. The source 505 of electromagnetic radiation can be, for example, a plurality of individual diode lasers, each coupled to an individual fiber optic 510.

FIG. 5B illustrates an exemplary fiber optic system 550 including a source 555 of electromagnetic radiation coupled to a coupler 560 through a connector 565. A plurality of fiber optics 570 are adapted to receive electromagnetic radiation from the source 555 through the coupler 560. The source 555 can include, for example, an individual diode laser, which forms a laser beam that is split by the coupler 560 to deliver approximately the same quality and quantity of electromagnetic radiation to each individual fiber optic 570.

The invention is not limited to the number and/or arrangement of fiber optics shown in FIGS. 5A-5B. Rather, a fiber optic system can be adapted for virtually any number and arrangement of fiber optics and/or needles. A fiber optic system can also be adapted for fiber optics of varying length. In various embodiments, the plurality of fiber optics receives a beam of radiation from a source of electromagnetic radiation. The apparatus can include a source of electromagnetic radiation. The source of electromagnetic radiation can be a laser, a light emitting diode, an incandescent lamp, a flash lamp, or a gas discharge lamp. Each fiber optic can employ free-space coupling to deliver electromagnetic radiation to treat the biological tissue. The beam of electromagnetic radiation can have a power between about 0.1 watts and about 500 watts. In one embodiment, the power delivered by each fiber optic is less than about 1 W. The beam of electromagnetic radiation can have a pulse duration between about 0.1 microseconds and about 10 seconds.

A fiber optic system can include a control system that can control the fiber optics individually. In one embodiment, the control system can deliver electromagnetic radiation to a subset of the fiber optics. The subset of fiber optics can match a pattern of a target, to treat the target and spare surrounding tissue. For example, the target can be a vein and the controller can deliver electromagnetic radiation to curvilinear array of fiber optics to treat the vein and to spare the tissue surrounding the vein. The control system can control the properties of electromagnetic radiation delivered to each fiber optic. For example, the fluence, wavelength, and/or duration of the electromagnetic radiation delivered to each fiber optic can be controlled.

In various embodiments, the beam of electromagnetic radiation can have a wavelength between about 400 nanometers and about 10,600 nanometers. The beam of electromagnetic radiation can have a wavelength between about 1195 and about 1235 nm and/or between about 1695 and about 1735 nm, which can be advantageous because these two wavelength regions are preferentially absorbed by fat relative to other chromophores such as water. A laser device can operate in the region from about 1.2 to about 1.7 microns, which is fat selective. Although fat-selective wavelengths can provide advantages such as selectivity, they are not always necessary because the electromagnetic radiation can be delivered directly to the fatty tissue. Wavelengths can also be selected to target water and/or other chromophores in fatty tissue. In various embodiments, fatty tissue can be irradiated at an infrared wavelength at which the ratio of absorption of the radiation by fatty tissue to absorption by water is 0.5 or greater, and preferably greater than one. In particular the electromagnetic radiation can be at a wavelength between about 880 to about 935 nm, about 1150 to about 1230 nm, about 1690 to about 1780 nm, and/or about 2250 to about 2450 nm with a fluence and a duration sufficient to treat fatty tissue. The electromagnetic radiation can have a wavelength between about 900 to about 930 nm, about 1190 to about 1220 nm, about 1700 to about 1730 nm, and/or about 2280 to about 2360 nm. The wavelength of approximately 920, 1210, 1715, and 2300 nm can be particularly effective. The wavelength can be selected to penetrate to a specific depth, for example, to about 1.2 microns. The fluence and duration of irradiation can vary depending upon the location and/or identity of the biological tissues being treated, the source of electromagnetic radiation, the wavelength(s), and the size of biological tissue. In various embodiments the treatment fluence can be, for example, approximately 0.5 J/cm² to 500 J/cm². Treatment parameters can be varied during a treatment and/or between fiber optics within the array. In certain embodiments, the invention can elevate the temperature of subcutaneous fat without substantially heating the dermis and/or epidermis.

A cooling system can be used to modulate the temperature in a region of biological tissue and/or minimize unwanted thermal injury to untargeted region of biological tissue. For example, the system can cool the skin before, during, or after delivery of radiation, or a combination of the aforementioned. Cooling can include contact conduction cooling, evaporative spray cooling, convective air flow cooling, or a combination of the aforementioned. In one embodiment, the handpiece includes a skin contacting portion that can be brought into contact with a region of skin. The base member can be cooled. A cooling plate can also be cooled. The cooling pale can be adjacent the base member. The cooling pale can define a plurality of holes through which the needles can pass. By cooling only a region of the target region or by cooling different regions of the target region to different extents, the degree of thermal injury of regions of the target region can be controlled.

In various embodiments, local anesthesia can be administered to the patient. Anesthesia can be delivered prior to and/or during delivering the beam of radiation or penetrating the biological tissue. In one embodiment, the anesthesia can be injected directly into the biological tissue. Anesthesia delivery can also include applying a topical anesthetic to the biological tissue. Alternatively, the method can include the use of general anesthesia. Performing the procedure without anesthesia can be beneficial for patients who may have an adverse reaction to anesthesia. Use of local anesthetic can also reduce cost of a procedure by eliminating the need for an anesthesiologist.

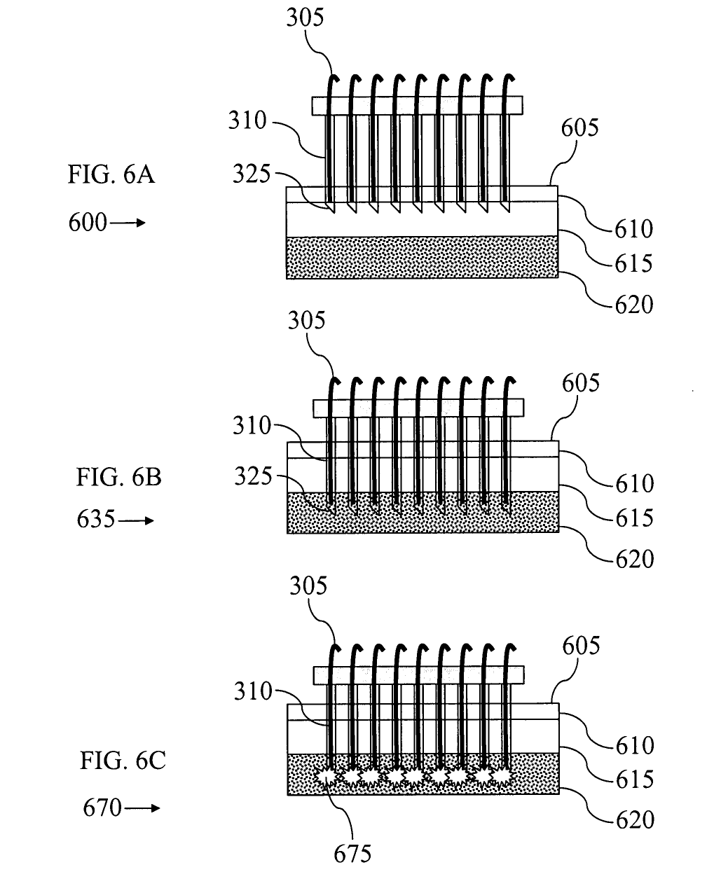

FIGS. 6A-6C illustrate a method for treating biological tissue. The biological tissue can be skin having a surface 605, an epidermis 610, a dermis 615, and subcutaneous fatty tissue 620. The subcutaneous fatty tissue 120 can include any of the features of the hypodermis.

In FIG. 6A, step 600 shows the plurality of needles 110 penetrating the surface 605 of the biological tissue. The plurality of needles 110 also penetrates the epidermis 610 and the dermis 615. Penetrating the surface 605 of the biological tissue with the plurality of needles 110 can form an angle of about 45 degrees between the surface 605 of the biological tissue and each needle. In various embodiments, penetrating the surface 605 of the biological tissue with the plurality of needles 110 forms an angle of about 30 degrees and about 90 degrees between the surface 605 of the biological tissue and each needle.

In FIG. 6B, step 635 shows the positioning of each end 125 within the subcutaneous fatty tissue 620. In some embodiments, the plurality of fiber optics 105 are positioned within the plurality of needles 110 after step 635. In other embodiments, the fiber optics 105 are positioned within the plurality of needles 110 prior to step 600, in which case the fiber optics 105 may require adjustment after step 635. The fiber optics 105 can be positioned within the plurality of needles 110 by a push switch mechanism. The fiber optics 105 can be disposable.

In FIG. 6C, step 670 shows the delivery of electromagnetic radiation 675 through the plurality of fiber optics 105 to the subcutaneous fatty tissue 620 to treat the biological tissue. The electromagnetic radiation 675 can melt and/or liquefy at least a portion of the subcutaneous fatty tissue 620. The method can include allowing the melted and/or liquefied fatty tissue to escape through the needle holes. The method can also include suctioning the melted and/or liquefied fatty tissue through the plurality of needles 110 and/or another means for suctioning. In some embodiments, the needle is partially retracted to expose at least a portion of the fiber optics 105 to the subcutaneous fatty tissue 620. The electromagnetic radiation 675 can also be delivered through one or more openings defined by the needle 110.

In various embodiments, the method can include the additional steps of (i) removing each end 125 from the subsurface volume 620 of the biological tissue; (ii) translating and/or rotating the plurality of needles 110 relative to the biological tissue; (iii) penetrating the surface 605 of the of biological tissue with the plurality of needles 310; (iv) positioning the each end 125 within a second subsurface volume (not shown) of the biological tissue; and (v) delivering electromagnetic radiation 675 through each fiber optic 105 inserted within the bore to the second subsurface volume of the biological tissue to treat the biological tissue. Translating or rotating the plurality of needles 110 relative to the biological tissue can form a larger area of coverage (e.g., positioning the each end 125 within a second subsurface volume) and/or higher coverage of a single area (e.g., repositioning each end 125 within a portion of the subsurface volume that was already treated).

In some embodiments, the method can include moving a portion of the at least one fiber optic within the subsurface volume of biological tissue while delivering electromagnetic radiation. For example, the plurality of needles 310 can be moved within the subsurface volume 620 of the biological tissue while delivering electromagnetic radiation 675 to maximize the amount of fatty tissue melted and/or liquefied. The melted and/or liquefied the fatty tissue can drain through the needle holes and/or be removed by suctioning. Suctioning can include removing the fiber optic 115 from at least a portion of the bore 120 and applying a vacuum 310. In one embodiment massage can be employed to aid in the removal of melted and/or liquefied fatty tissue.

In certain embodiments, the method can include mitigating pain and/or discomfort. For example, anesthesia can be administered before step 600 when the plurality of needles 110 penetrates the surface 605 of the biological tissue or after step 600. Anesthesia can also be administered during the treatment.

In various embodiments, the method can include cooling at least a portion of the biological tissue, to mitigate undesired thermal damage to the portion of the biological tissue. For example, the epidermis and/or dermis can be cooled in conjunction with delivering increased fluences of electromagnetic radiation to the subcutaneous fatty tissue to mitigate undesired thermal damage to the epidermis and/or dermis while increasing the efficacy of treatment of the subcutaneous fatty tissue. A member can apply pressure to and/or cool the skin, to displace blood from a region of biological tissue, to limit damage to blood vessels in the region of biological tissue.

In one embodiment, the method includes contacting the skin with a cooled plate to cool and numb the skin. The plate can define a plurality of holes. A plurality of needles 110 can penetrate the surface 605 of the biological tissue through the plurality of holes in the plate. Alternatively, the plate can be removed before the plurality of needles 110 penetrate the surface 605 of the biological tissue.

The treatment radiation can damage one or more fat cells so that at least a portion of lipid contained within can escape and/or can be drained from the treated region. At least a portion of the lipid can be carried away from the tissue through a biological process. In one embodiment, the body's lymphatic system can drain the treated fatty tissue from the treated region. In an embodiment where a fat cell is damaged, the fat cell can be viable after treatment. In one embodiment, the treatment radiation can destroy one or more fat cells. In one embodiment, a first portion of the fat cells is damaged and a second portion is destroyed. In one embodiment, a portion of the fat cells can be removed to selectively change the shape of the body region.

In some embodiments, the beam of radiation can be delivered to the target region to thermally injure, damage, and/or destroy one or more fat cells. For example, the beam of radiation can be delivered to a target chromophore in the target region. Suitable target chromophores include, but are not limited to, a fat cell, lipid contained within a fat cell, fatty tissue, a wall of a fat cell, water in a fat cell, and water in tissue surrounding a fat cell. The energy absorbed by the chromophore can be transferred to the fat cell to damage or destroy the fat cell. For example, thermal energy absorbed by dermal tissue can be transferred to the fatty tissue. In one embodiment, the beam of radiation is delivered to water within or in the vicinity of a fat cell in the target region to thermally injure the fat cell.

In various embodiments, treatment radiation can affect one or more fat cells and can cause sufficient thermal injury in the dermal region of the skin to elicit a healing response to cause the skin to remodel itself. This can result in more youthful looking skin and an improvement in the appearance of cellulite. In one embodiment, sufficient thermal injury induces fibrosis of the dermal layer, fibrosis on a subcutaneous fat region, or fibrosis in or proximate to the dermal interface. In one embodiment, the treatment radiation can partially denature collagen fibers in the target region. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target region can change and/or improve the appearance of the skin of the target region, as well as thicken the skin, tighten the skin, improve skin laxity, and/or reduce discoloration of the skin.

In various embodiments, a zone of thermal injury can be formed at or proximate to the dermal interface. Fatty tissue has a specific heat that is lower than that of surrounding tissue (fatty tissue, so as the target region of skin is irradiated, the temperature of the fatty tissue exceeds the temperature of overlying and/or surrounding dermal or epidermal tissue. For example, the fatty tissue has a volumetric specific heat of about 1.8 J/cm³ K, whereas skin has a volumetric specific heat of about 4.3 J/cm³ K. In one embodiment, the peak temperature of the tissue can be caused to form at or proximate to the dermal interface. For example, a predetermined wavelength, fluence, pulse duration, and cooling parameters can be selected to position the peak of the zone of thermal injury at or proximate to the dermal interface. This can result in collagen being formed at the bottom of the dermis and/or fibrosis at or proximate to the dermal interface. As a result, the dermal interface can be strengthened against fat herniation. For example, strengthening the dermis can result in long-term improvement of the appearance of the skin since new fat being formed or untreated fat proximate the dermal interface can be prevented and/or precluded from crossing the dermal interface into the dermis.

In one embodiment, fatty tissue is heated by absorption of radiation, and heat can be conducted into dermal tissue proximate the fatty tissue. The fatty tissue can be disposed in the dermal tissue and/or can be disposed proximate to the dermal interface. A portion of the dermal tissue (e.g., collagen) can be partially denatured or can suffer another form of thermal injury, and the dermal tissue can be thickened and/or be strengthened as a result of the resulting healing process. In such an embodiment, a fat-selective wavelength of radiation can be used.

In one embodiment, water in the dermal tissue is heated by absorption of radiation. The dermal tissue can have disposed therein fatty tissue and/or can be overlying fatty tissue. A portion of the dermal tissue (e.g., collagen) can be partially denatured or can suffer another form of thermal injury, and the dermal tissue can be thickened and/or be strengthened as a result of the resulting healing process. A portion of the heat can be transferred to the fatty tissue, which can be affected. In one embodiment, water in the fatty tissue absorbs radiation directly and the tissue is affected by heat. In such embodiments, a water selective wavelength of radiation can be used.

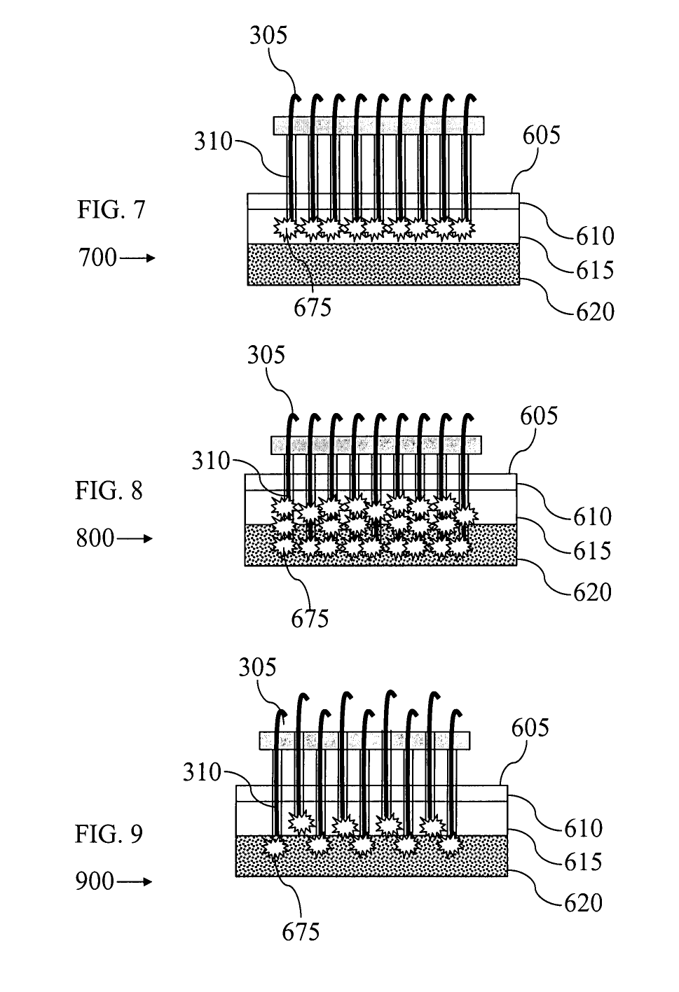

In FIG. 7, step 700 shows the delivery of electromagnetic radiation 675 through the plurality of fiber optics 105 to the dermis 615 to treat the biological tissue. The electromagnetic radiation 675 can denature collagen and/or otherwise injure at least a portion of the dermis 615. In various embodiments, step 700 can precede and/or follow step 670 shown in FIG. 6C. Step 700 can be a discreet step (e.g., position the plurality of fiber optics 105 within the dermis 615 and deliver electromagnetic radiation 675) or continuous (e.g., deliver electromagnetic radiation 675 while the fiber optics 105 are being inserted and/or withdrawn from the biological tissue).

In FIG. 8, step 800 shows the simultaneous delivery of electromagnetic radiation 675 through the plurality of fiber optics 105 to the dermis 615 and to the subcutaneous fatty tissue 620. The method of step 800 can be achieved by employing a needle 110 defining one or more openings that allow electromagnetic radiation to radiate from a region other than about the end like, for example, the needle 110 shown in FIG. 1C. The amount of electromagnetic radiation directed to a specific depth can be controlled by the number, size, and/or transmittance of the openings. The needle 110 can be positioned within the biological tissue and the intensity and/or duration of electromagnetic radiation directed to a specific depth can be controlled by the rate of insertion and/or withdrawal of the fiber optics 105.

In various embodiments, such as step 800, the electromagnetic radiation is delivered approximately perpendicular to the axis of the needle 110. In some embodiments, the needles are spaced such that the electromagnetic radiation forms zones of thermal injury separated by substantially undamaged biological tissue.

In FIG. 9, step 900 shows the simultaneous delivery of electromagnetic radiation 675 through the plurality of fiber optics 105 to the dermis 615 and to the subcutaneous fatty tissue 620. The method of step 900 can be achieved by employing a plurality of needles 110 of varying length. For example, one or more needles 110 can be within the dermis 615 and one or more needles 110 can be within the subcutaneous fatty tissue 620. In other examples, needles 110 of varying length can be used for simultaneous delivery of electromagnetic radiation 675 to varying depths of the dermis 615 and/or subcutaneous fatty tissue 620.

The methods shown in FIGS. 6-9 can include the advantages of even heating of the biological tissue, delivery of electromagnetic radiation directly to the subsurface volume of biological tissue being targeted, and/or reducing trauma from the treatment. In various embodiments, the method can form a pattern of thermal injury within the biological tissue.

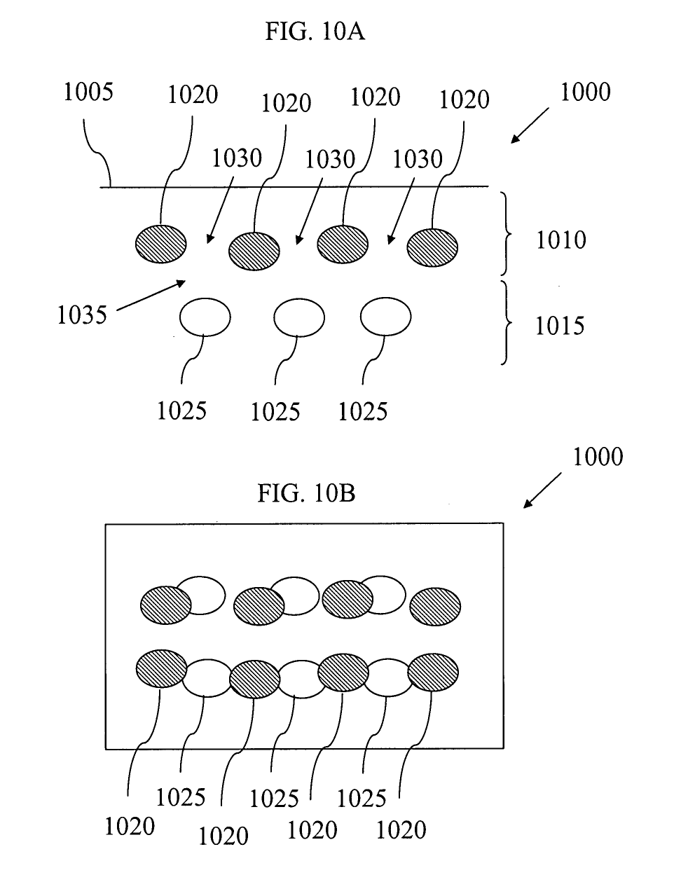

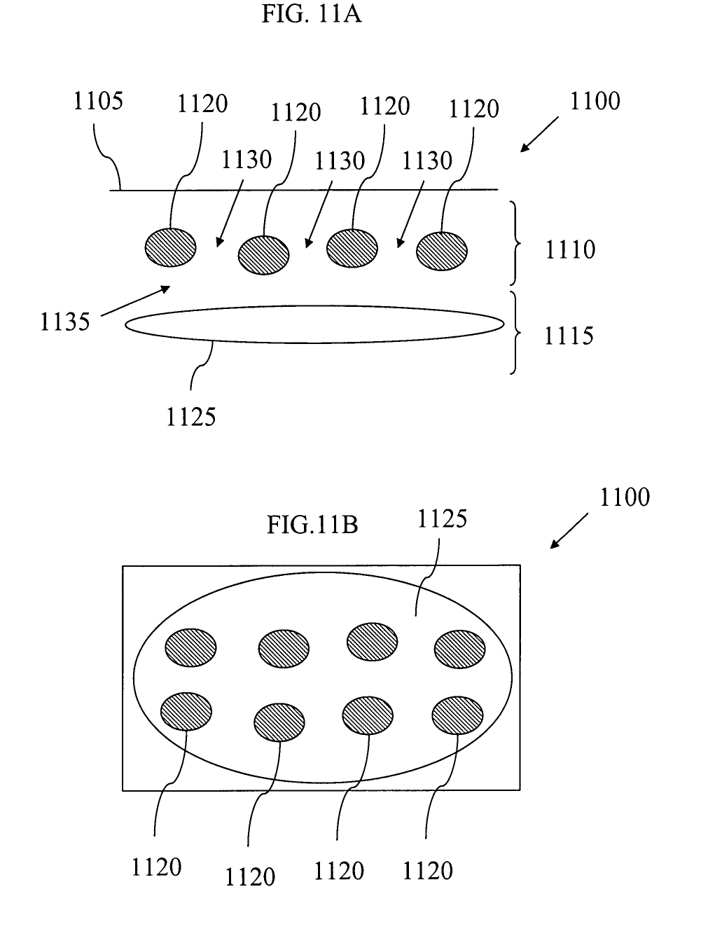

FIG. 10A shows a cross-section of an exemplary region of skin 1000 including a skin surface 1005, a first region 1010 of skin at a first depth, a second region 1015 of skin at a second depth, a plurality of first thermal injuries 1020 in the first region 1010, and a plurality of second thermal injuries 1025 in the second region 1015. Each plurality of thermal injuries can be separated by substantially undamaged skin 1030. The thermal injuries at the first depth can be separated from the thermal injuries at the second depth by an intermediate region of substantially undamaged skin 1035.

The first thermal injuries 1020 can be more or less severe than the second thermal injuries 1025. For example, the first thermal injuries 1020 can denature collagen within the dermis and the second thermal injuries 1025 can be necrotic thermal injuries within the subcutaneous fatty tissue. Necrotic thermal injuries can melt and/or liquefy the fatty tissue. Necrotic thermal injuries can elicit a healing response from the skin. Denaturing collagen can accelerate collagen synthesis, tighten skin, mitigate wrinkles, and/or elicit a healing response. An interspersed plurality of first thermal injuries 1020 and second thermal injuries 1025 can intensify the skin's healing response and accelerate recovery and healing, as compared to a large, continuous thermal injury. Healing can initiate from less injured or substantially undamaged skin 1030 adjacent the plurality of first thermal injuries 1020 and/or second thermal injuries 1025.

FIG. 10B shows a top view of the region of skin 1000 shown in FIG. 10A. The first and second thermal injuries can form less than about 100% coverage of a target region of skin, which can be measured as the area corresponding to the thermal injuries as seen from the skin surface. In some embodiments, the first and second thermal injuries can form about 100% coverage of a target region of skin.

FIG. 11A shows a cross-section of an exemplary region of skin 1100 including a skin surface 1105, a first region 1110 of skin at a first depth, a second region 1115 of skin at a second depth, a plurality of first thermal injuries 1120 in the first region 1110, and a second thermal injury 1125 in the second region 1115. Each of the plurality of first thermal injuries 1120 can be separated by substantially undamaged skin 1130. The first thermal injuries 1120 at the first depth can be separated from the second thermal injury 1125 by an intermediate region of substantially undamaged skin 1135.

The first thermal injuries 1120 can be more or less severe than the second thermal injury 1125. For example, the first thermal injuries 1120 can denature collagen within the dermis and the second thermal injury 1125 can be necrotic thermal injury within the subcutaneous fatty tissue. Necrotic thermal injuries can melt and/or liquefy the fatty tissue. Denaturing collagen can accelerate collagen synthesis, tighten skin, mitigate wrinkles, and/or elicit a healing response. The first thermal injuries 1120 overlying a second thermal injury 1125 can intensify the skin's healing response and accelerate recovery and healing, as compared to a large, continuous, severe thermal injury. Healing can initiate from less injured or substantially undamaged skin 1130 adjacent the plurality of first thermal injuries 1120 and/or second thermal injury 1125.

FIG. 11B shows a top view of the region of skin 1100 shown in FIG. 1A. The first and second thermal injuries can form about 100% coverage of a target region of skin, which can be measured as the area corresponding to the thermal injuries as seen from the skin surface. In some embodiments, the first and second thermal injuries can form less than about 100% coverage of a target region of skin.

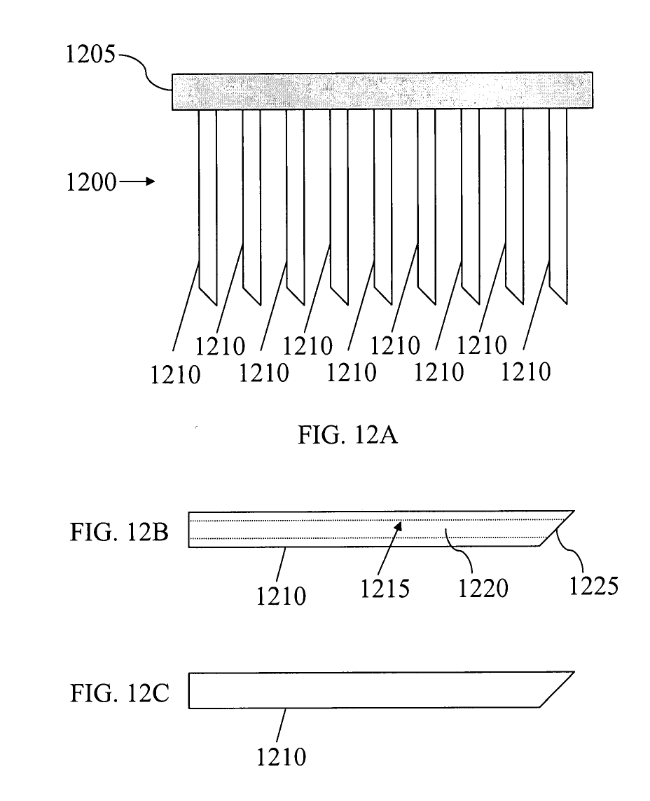

FIG. 12A illustrates an apparatus 1200 for treating biological tissue including a plurality of waveguides 1210 extending from a base member 1205. The base member 1205 can be made from a metal, plastic, or polymer material. The plurality of waveguides 1210 can be attached to the base member 1205, or can be removable. The base member 1205 can be flexible, which can allow the plurality of waveguides 1210 extending from the base member 1205 to match a contour of the biological tissue.

FIG. 12B illustrates one embodiment of a waveguide 1210 in detail. The waveguide 1210 can be a hollow waveguide. For example, the waveguide 1210 can be a needle that defines a bore 1220 and has an end 1225. In some embodiments, at least a portion of an inner surface 1215 has a coating to facilitate transmission of the electromagnetic radiation. The inner surface 1215 can be covered with a single or multilayer film, which for example, guides light by Bragg reflection (e.g., a photonic-crystal fiber). The film can be silver. Small prisms around the waveguide, which reflect light via total internal reflection, can be used. In other embodiments, the inner surface 1215 is not coated and can be polished metal. In various embodiments, the hollow waveguide 1210 can include silica, glass, sapphire, crystal, metal, and/or plastic materials. The hollow waveguide 1210 can be a naked waveguide or can be a waveguide inserted into the bore 120 of a needle 110. In various embodiments, the hollow waveguide 1210 can define one or more openings (not shown) that allow electromagnetic radiation to radiate from the waveguide 1210 from a region other than about the end 1225. The one or more openings can facilitate simultaneous treatment at more than one depth within the biological tissue.

FIG. 12C illustrates another embodiment of a waveguide 1210 in detail. The waveguide 1210 can be a solid waveguide including silica, glass, sapphire, crystal, metal, and/or plastic materials. The waveguide 1210 can include one or more layers and/or coatings to facilitate transmission of the electromagnetic radiation. In some embodiments, a rigid, solid waveguide 1210 is adapted to penetrate biological tissue. In other embodiments, a solid waveguide 1210 is inserted into the bore 120 of a needle 110. A waveguide can have similar dimensions to the needles and fiber optics described above. Each waveguide 1210 can be disposable. The base member 1205 can be disposable. In one embodiment, the base member 1205 and plurality of waveguides 1210 can be a disposable, and can be in the form of a cartridge. Alternatively, the waveguide 1210 and/or base member 1205 can be sterilized and reusable. The plurality of waveguides 1210 form an array capable of penetrating a biological tissue and positioning each end 1225 within a subsurface volume of the biological tissue. Each waveguide 1210 is capable of delivering electromagnetic radiation to the subsurface volume of the biological tissue to treat the biological tissue. A vacuum can be applied to the subsurface volume of biological tissue through a hollow waveguide 1210. Alternatively, the hollow and/or solid waveguide can be retracted from at least a portion of the bore 120 of a needle 110, and a vacuum can be applied to the subsurface volume of biological tissue through the needle 110. In some embodiments an apparatus can include one or more waveguides for delivering electromagnetic radiation, and one or more needles for applying a vacuum, to the subsurface volume of the biological tissue. In various embodiments, any of the needle and fiber optic features and methods described herein can be used with waveguides 1210.

In some embodiments, the biological tissue can be covered with an absorbent material to draw one or more fluids from the biological tissue. The absorbent material can be a wound dressing that includes a substance to draw fluid from the biological tissue to increase the biological tissue's response to the injury, remove unwanted or damaged biological tissue, and/or to induce shrinkage of the biological tissue.

Skin shrinkage can result in an improvement in the skin's appearance. For example, puncturing and treating the skin with radiation can damage or destroy fatty tissue, and can elicit a healing response to cause the skin to remodel itself. Skin shrinkage can thicken the skin, tighten the skin, improve skin laxity, induce collagen formation, promote fibrosis of the dermal layer, and result in rejuvenation of the skin. In certain embodiments, improvement occurs in the dermal region of the skin. Furthermore, a treatment can include a series of treatment cycles, so that skin can be reduced gradually, and/or the skin can be tightened gradually, resulting in a more cosmetically appealing appearance.

The skin can shrink by a range of a factor of about 1 to about 10. In certain embodiments, the skin can shrink by at least a factor of about 1.25 to about 5. In some embodiments, the skin can shrink by at least a factor of about 1.1, 2, 3, or 4. Skin shrinkage can be measured by determining the percentage decrease in a volume of target tissue. Skin shrinkage can be measured by determining the percentage decrease in the surface area of the target tissue.

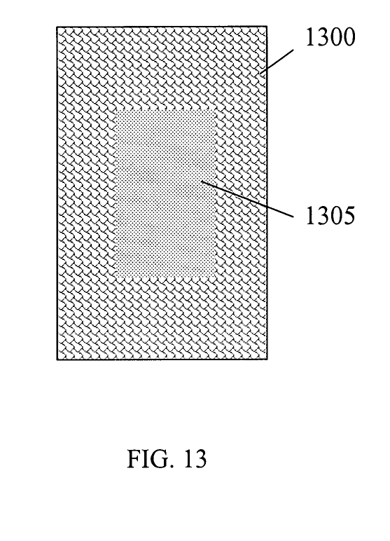

FIG. 13 shows an absorbent pad 1300 including an absorbent material 1305 disposed on the absorbent pad 1300. In certain embodiments, the absorbent pad 1300 alone is the absorbent material. A dressing can be applied to the target region of skin. The dressing can include the absorbent pad 1300 and the absorbent material 1305.

The absorbent material 1305 can draw fluid from the skin. The fluid can be one or more of a body fluid, a cellular fluid, melted and/or liquefied fatty tissue, or water. The absorbent material 1305 can include a solid or a liquid. The absorbent material 1305 can include salt or glycerol. For example, the absorbent material 1305 can include at least one of a salt mixture or a composition including a salt. The absorbent material 1305 can be a desiccating agent, a solution adapted to draw a body fluid from the target region, or a solution adapted to draw a cellular fluid from the target region. The absorbent material can include an antiseptic, an antibiotic, and/or a disinfectant.

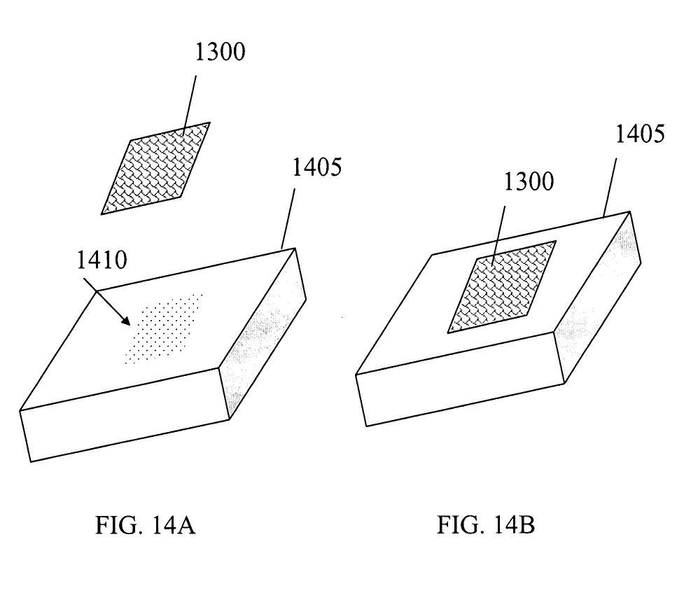

FIG. 14A shows a region of skin 1405 treated with a puncturing device to cause a plurality of puncture marks 1410. FIG. 14B shows the absorbent pad 1300 covering the region of skin 1405. In some embodiments, the absorbent pad 1300 or material 1305 is applied for a period of between about 1 minute and about 3 days. Depending on the treatment, longer and shorter time frames can be used. The absorbent pad 1300 or material 1305 can be applied for a period of at least 1 minute. In some embodiments, the absorbent pad 1300 or material 1305 can be applied for about 1 minute, about 15 minutes, about 30 minutes, about 60 minutes, about 2 hours, about 6 hours, about 12 hours, about 1 day, about 2 days, or about 3 days. In certain embodiments, a first pad can be removed from the skin and a second pad can be applied.

The absorbent pad 1300 or the absorbent material 1305 can cause the fluid to migrate from the target region of skin to the absorbent material 1305. For example, the fluid can migrate to an outer surface of the skin so the absorbent material 1305 can absorb the fluid.

The severity of the treatment can be varied, for example, by varying the density of skin punctures, the size of the needles, the depth of the punctures, and by varying the concentration of the topical agents used. More aggressive treatment may lead to beneficial skin shrinkage with a scar. Less aggressive treatments may produce beneficial skin shrinkage without producing a scar.

In certain embodiments, the absorbent material 1305 can be applied directly to the skin 1405. A bandage, e.g., the absorbent pad 1300, can be applied over the skin 1405 and the absorbent material 1305.

In certain embodiments, suction can be used to remove fluid from the biological tissue. For example, as the needles are removed from the biological tissue, the force of withdraw can draw fluid to the surface of the biological tissue. In some embodiment, a suction system or syringe is used.

In certain embodiments, the biological tissue can be irrigated after the biological tissue is punctured. This can include using a needle or syringe to inject a fluid into the biological tissue.

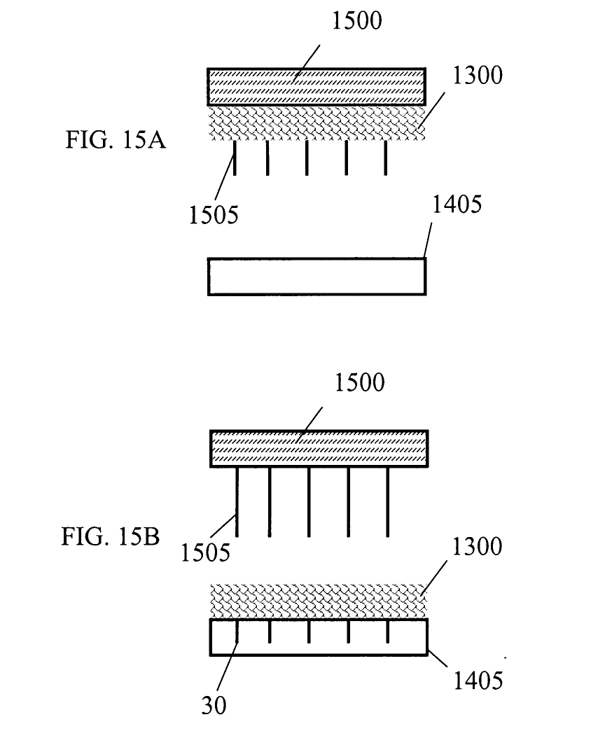

FIG. 15A shows an embodiment where the absorbent pad 1300 is affixed to a base member 1500. The base member 1500 is placed proximate to the skin 1405 so the needles 1505 can puncture the skin 1405. Referring to FIG. 15B, with base member 1500 withdrawn, the absorbent pad 1300 is ejected from the base member 1500 and the absorbent pad 1300 covers the skin, including the puncture marks 1510 remaining in the skin from the needles 1505. The absorbent pad 1300 can remove fluid from the skin 1405 to cause the skin to shrink 1405.

In certain embodiments, a beam of radiation can be applied through the surface of the biological tissue to affect the biological tissue. The beam of radiation can augment or complement the treatment using the waveguides or needles. The beam of radiation can be applied before, during, or after insertion of the waveguides or needles. For example, the beam of radiation can be delivered to the target region to thermally injure, damage, and/or destroy one or more fat cells. This can lead to reshaping of the biological tissue region as the skin size is reduced. The surface of the biological tissue can be cooled to protect overlying tissue.

In some embodiments, the beam of radiation can cause sufficient thermal injury in the dermal region of the skin to elicit a healing response to cause the skin to remodel itself. This can result in more youthful looking skin. In one embodiment, sufficient thermal injury induces fibrosis of the dermal layer, fibrosis on a subcutaneous fat region, or fibrosis in or proximate to the dermal interface. In one embodiment, the treatment radiation can partially denature collagen fibers in the target region. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target region can change and/or improve the appearance of the skin of the target region, as well as thicken the skin, tighten the skin, improve skin laxity, and/or reduce discoloration of the skin.

In some embodiments, the beam of radiation can be used to treat acne, erythema, oily skin, pigmented lesions, pores, scarring, vascular lesions (including port wine stains), hair removal, and hair regrowth.

While the invention has been particularly shown and described with reference to specific embodiments, it should be understood by those skilled in the art that various changes in form and detail may be made without departing from the spirit and scope of the invention as defined by the appended claims.

1. An apparatus for treating biological tissue comprising:
a base member;
a plurality of needles extending from the base member, each needle defining a bore capable of receiving a fiber optic and having an end, the plurality of needles forming an array capable of penetrating a biological tissue and positioning each end within a subsurface volume of the biological tissue; and
a plurality of fiber optics, each fiber optic adapted for insertion into the bore of each needle, and each fiber optic capable of delivering electromagnetic radiation to the subsurface volume of the biological tissue to treat the biological tissue.

2. The apparatus of claim 1 wherein a laser, a light emitting diode, a flash lamp, or a gas discharge lamp provides a beam of electromagnetic radiation to be delivered by each fiber optic.

3. The apparatus of claim 1 wherein the fiber optic is adapted to be movable within the bore, extendable beyond the end, and retractable into the bore.

4. The apparatus of claim 1 wherein the fiber optic comprises sapphire.

5. The apparatus of claim 1 wherein a wavelength between about 400 nanometers and about 10,600 nanometers characterizes a beam of electromagnetic radiation to be delivered by each fiber optic.

6. The apparatus of claim I wherein each needle is adapted for penetrating biological tissue to a depth of about 1.5 to about 30 mm from a surface of the biological tissue.

7. The apparatus of claim 1 wherein a diameter of less than about 1 millimeter characterizes each needle.

8. The apparatus of claim 1 wherein a diameter between about 0.2 mm and about 2 mm characterizes each needle.

9. The apparatus of claim 1 wherein each fiber optic employs free-space coupling to deliver electromagnetic radiation to treat the biological tissue.

10. The apparatus of claim 1 wherein a power between about 0.1 watts and about 500 watts characterizes a beam of electromagnetic radiation to be delivered by each fiber optic.

11. The apparatus of claim 1 wherein a pulse duration between about 0.1 microseconds and about 10 seconds characterizes a beam of electromagnetic radiation to be delivered by each fiber optic.

12. The apparatus of claim 1 further comprising a means for suctioning at least a portion of the biological tissue.

13. The apparatus of claim 1 further comprising a means for cooling at least a portion of the biological tissue.

14. The apparatus of claim 1 further comprising a means for mitigating pain in at least a portion of the biological tissue.

15. The apparatus of claim 1 further comprising a scanner for translating or rotating the base member.

16. The apparatus of claim 1 further comprising a source of a beam of electromagnetic radiation.

17. An apparatus for treating biological tissue comprising:
a base member;
a first needle extending from the base member, the first needle defining a first bore and having a first end;
a second needle extending from the base member and spaced from the first needle, the second needle defining a second bore and having a second end, the first needle and the second needle forming an array of needles capable of penetrating a biological tissue and positioning the first end and the second end within a subsurface volume of the biological tissue;
a first fiber optic adapted for insertion into the first bore; and
a second fiber optic adapted for insertion into the second bore, the first fiber optic and the second fiber optic capable of delivering electromagnetic radiation to the subsurface volume of the biological tissue to treat the biological tissue.

18. A method for treating biological tissue comprising:
penetrating a surface of a biological tissue with a plurality of needles, each needle defining a bore capable of receiving a fiber optic and having an end;
positioning each end within a subsurface volume of the biological tissue; and
delivering electromagnetic radiation through a plurality of fiber optics, at least one fiber optic of the plurality of fiber optics inserted within the bore of each needle, to the subsurface volume of the biological tissue to treat the biological tissue.

19. The method of claim 18 wherein penetrating the surface of the biological tissue with the plurality of needles forms an angle of about 45 degrees and about 90 degrees between the surface of the biological tissue and each needle.

20. The method of claim 18 further comprising moving a portion of the at least one fiber optic within the subsurface volume of biological tissue while delivering electromagnetic radiation.

21. The method of claim 18 further comprising applying suction to the subsurface volume of the biological tissue.

22. The method of claim 18 further comprising cooling at least a portion of the biological tissue.

23. The method of claim 18 further comprising mitigating at least a portion of pain or discomfort related to the method.

24. The method of claim 18 further comprising:
removing each end from the subsurface volume of the biological tissue;
translating or rotating the plurality of needles relative to the biological tissue;
penetrating the surface of the of biological tissue with the plurality of needles;
positioning the each end within a second subsurface volume of the biological tissue; and
delivering electromagnetic radiation through each fiber optic inserted within the bore to the second subsurface volume of the biological tissue to treat the biological tissue.

25. The method of claim 18 further comprising inserting each fiber optic in the bore.

26. A method for treating a biological tissue comprising:
penetrating a surface of a biological tissue with a plurality of waveguides, each waveguide having an end;
positioning each end within a subsurface volume of the biological tissue; and
delivering electromagnetic radiation through the plurality of waveguides to the subsurface volume of the biological tissue to treat the biological tissue.

27. An apparatus for treating biological tissue comprising a plurality of waveguides extending from a base member, each waveguide having an end, the plurality of waveguides forming an array capable of penetrating a biological tissue and positioning each end within a subsurface volume of the biological tissue and delivering electromagnetic radiation to the subsurface volume of the biological tissue to treat the biological tissue.
